(19) United States
(12) Patent Application Publication
(10) Pub. No.: US 2022/0175717 A1
(43) Pub. Date: Jun. 9, 2022

(54) PHENPROCOUMON TTS

(71) Applicant: LTS LOHMANN THERAPIE-SYSTEME AG, Andemach (DE)

(72) Inventors: Dirk TRENZ, Lahnstein (DE); Thomas HILLE, Neuwied (DE)

(21) Appl. No.: 17/438,802

(22) PCT Filed: Feb. 6, 2020
(86) PCT No.: PCT/EP2020/052919

§ 371 Date: Sep. 13, 2021

(30) Foreign Application Priority Data
Mar. 14, 2019 (DE) 10 2019 106 532.1

(51) Int. Cl. A61K 31/352; A61K 9/00; A61K 9/70

(52) U.S. Cl. CPC A61K 31/352; A61K 9/7061; A61K 9/0014

(57) ABSTRACT

The present invention relates to a transdermal therapeutic system for the cutaneous administration of phenprocoumon, comprising an active-substance-impermeable backing layer, an adhesive matrix layer and optionally a removable protective layer, the adhesive matrix layer containing phenprocoumon and at least one matrix polymer, and the content of phenprocoumon in the matrix polymer being ≤7.5 wt. %. By virtue of the low load, it is ensured that the system releases the active ingredient substantially in high release rates, because high thermodynamic activity of the active ingredient is achieved. The present invention also relates to a method for producing a corresponding transdermal therapeutic system.

The present invention relates to a transdermal therapeutic system for the administration of phenprocoumon, comprising an active-substance-impermeable backing layer, an adhesive matrix layer and an optionally removable protective layer, and to a method for producing such a system.

Phenprocoumon, also known under the trade names Marcumar and Falithrom, is a chemical compound from the group of 4-hydroxycoumarins and is used as a drug to inhibit plasma blood clotting (anticoagulation).

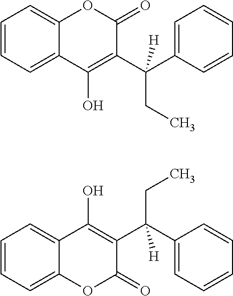 [SMILES: CC[C@H](c1ccccc1)c1c(O)c2ccccc2oc1=O ; CC[C@@H](c1ccccc1)c1c(O)c2ccccc2oc1=O]

It is evident from the shown formula of phenprocoumon that the stereocentre in phenprocoumon is positioned directly adjacent to an enol group, so that the two enantiomers can merge into each other via a keto-enol tautomerism. The enol form additionally represents a vinylogous carboxylic acid, which results in good solubility of phenprocoumon in acidic polyacrylates.

Phenprocoumon is used within the scope of long-term thrombosis prophylaxis or recurrence prophylaxis (following heparin therapy or alternatively in the case of heparin intolerance), after the implantation of artificial heart valves/artificial vascular bypasses (fern-pop/Y-prosthesis, etc.), heart support systems (assist devices) or in the case of atrial fibrillation, in order to prevent thrombus formation and consequent embolisms.

During the adjustment phase with tablets (3 mg each) of about 5 to 7 days, the patient's blood is taken every 2^{nd} to 3^{rd} day, in the event of a stable response every 2 to 4 weeks, for control and then the patient's next phenprocoumon dosage is determined. If the patient's response is stable and he/she is suitable (he/she must be physically and mentally capable), he/she can carry out the regular control measurement and adjustment of the medicament dose himself/herself after having received training (coagulation self-management). The procedure and the measuring devices to be used for this are similar to the known sugar self-tests.

The oral intake of phenprocoumon is unfortunately associated with significant disadvantages, as the absorption is more dependent on the patient's food intake than with other drugs. Especially, the effect of phenprocoumon is influenced by the vitamin K content of the food. For a constant effect, it is therefore important that the patient only eats small amounts of foods rich in vitamin K. Even taking this restriction into account, however, only about 70% of the values are in the therapeutic range.

Furthermore, there is a risk of phenprocoumon overdoses, since phenprocoumon, once absorbed by the body, is only broken down very slowly due to its extremely long biological half-life of up to 160 (!) hours. A continuous infusion of phenprocoumon would therefore actually be the drug form of choice, as the absorption of the active substance can be controlled and interrupted at any time via this type of administration. However, an obvious disadvantage of this form of administration is that a continuous infusion can be carried out only in hospital, but not within the scope of home care.

Accordingly, it was the aim of the present invention to provide an administration form of phenprocoumon which avoids the disadvantages of oral and intravenous administration described above.

Transdermal therapeutic systems (TTS) have become widely used in recent years as a form of administration for the treatment of numerous diseases, as these systems have many advantages over conventional administration forms. These include precise and constant active substance delivery, which allows the setting of a constant concentration of the active substance in the blood plasma. In addition, the first-pass effect can be circumvented and compliance (i.e. the patient's adherence to a treatment plan) can be improved, as the patient does not have to take tablets regularly. One advantage of transdermal therapeutic systems over other topical administration systems such as ointments or creams is that they can be applied to the exact area and thus in the exact dose, and there is no risk of accidentally wiping off an ointment and contaminating other skin sites. In addition, ointments or tablets have to be applied or administered regularly, since a sustained release of the active substance is usually not possible.

A few years ago, it was assumed that the implementation of active substances in transdermal therapeutic systems was generally unproblematic, so that this form of application could be used for a large variety of active substances. However, this assumption has turned out to be a fallacy in recent years, because the molecular transport of active substances via the skin is a limiting factor. For example, transport via the outer skin layer of the stratum corneum has proven to be too slow for many active substances, so that an effective delivery and thus an effective concentration of the active substance in the blood plasma could not be realised. On a commercial level, the delivery of active substances via transdermal therapeutic systems is therefore limited to few very potent active substances. An overview of this can be found, for example, in Wiedersberg et al, J. Controlled Release, 190 (2014), pp. 150-156.

Another disadvantage of TTS is that the active substance content and delivery rate are not identical.

At first glance, a TTS does not appear to be suitable for the administration of phenprocoumon, as the active substance is generally not an ideal candidate for a TTS due to the high dose required of up to 3 mg per day and its high lipophilicity. For this reason, no TTS with phenprocoumon has been developed or even approved to date.

A high active substance content in a transdermal therapeutic system is associated with the disadvantages that more active substance is required during production and that the used patch still contains a relatively large amount of active substance that must be disposed of. In addition, it is sought to avoid high active substance contents in transdermal therapeutic systems also for reasons of drug safety. A transdermal therapeutic system with a low active substance content would therefore be more economical, environmentally friendlier, and safer.

It is therefore also an aim of the invention to propose a transdermal therapeutic system for the delivery of phenprocoumon, the active substance content of which is low and in which phenprocoumon is released as far as possible from the TTS over the intended delivery period. The solution is all the more difficult because although both phenprocoumon and TTS have been known for a long time, no TTS has yet been described for the delivery of phenprocoumon. The reason for this is that the daily dose of 3 mg is considered by average experts to be too high for transdermal administration, i.e. higher than would permit development of a TTS of still acceptable size, i.e. having an area <50 cm². The good solubility of phenprocoumon in the conventional pressure-sensitive adhesives could also be a reason why a TTS for the therapeutic delivery of phenprocoumon TTS is not yet available, although there is a need for such a dosage form.

The above aim is addressed in accordance with the invention by a transdermal therapeutic system according to claim 1, which has an active-substance-impermeable backing layer, an adhesive matrix layer and optionally a removable protective layer, the adhesive matrix layer containing phenprocoumon and at least one matrix polymer, and the content of phenprocoumon in the matrix polymer being ≤12% by weight (specified as active substance content in the matrix polymer).

The active substance is preferably predominantly dissolved in the matrix polymer, i.e. at least 60% by weight, preferably at least 80% by weight, more preferably at least 90% by weight, and even more preferably at least 95% by weight of the active substance is dissolved in the matrix polymer. A larger proportion of crystallised active substance is disadvantageous, since in this case the active substance must first dissolve again for delivery to the skin, which impairs or prevents the establishment of a constant delivery profile.

Also preferred in the context of the invention described herein is that the matrix polymer has a saturation concentration Cₛ (to be determined according to the method described in the examples) in the range of about 0.3 to 10%, preferably in the range of 1 to 8%, and further preferably in the range of 2.5 to 7.5%.

The saturation concentration of the vinylogous acid phenprocoumon is lower in a matrix polymer without free carboxylic acid groups and/or carboxylate groups than in a neutral matrix polymer or a polymer without functional groups. Thus, at a lower active substance content it is possible to achieve a thermodynamic activity similar to that when phenprocoumon is used in high proportions.

It has also surprisingly been found that the transdermal therapeutic system according to the invention has good to sufficient adhesive strength, although the use of matrix polymers and especially polyacrylates with carboxy groups, which impart a high inherent tack to the polymer, is omitted. This embodiment is all the more astonishing because phenprocoumon has no inherent tack.

In a preferred embodiment, the transdermal therapeutic system contains in the matrix polymer 0.5 to 12% by weight, preferably 1 to 10% by weight, more preferably 2 to 7.5% by weight and even more preferably 2.5 to 7.3% by weight phenprocoumon, preferably in dissolved form.

A low phenprocoumon content nevertheless allows the desired daily dose to be reached in many cases. Advantages resulting from this are low active substance requirement for the production of the TTS, the resultant lower production costs, simpler disposal, and greater safety. In addition to the active substance phenprocoumon, the TTS according to the invention preferably does not contain any other active substances in pharmaceutically effective dosages.

With respect to the matrix polymer, the transdermal therapeutic system of the present invention is not subject to any relevant limitations. In the context of the present invention, the term “free carboxylic acid and/or carboxylate groups” means —CO₂H and —CO₂ groups which are present in unbound and non-complexed form. —CO₂ groups which are bound in the form of esters or which coordinate to complexing metals, especially transition metals such as titanium, are not to be regarded as free carboxylic acid and/or carboxylate groups, whereas carboxylate salts with non-coordinating metal ions, such as alkali metal ions or alkaline earth metal ions, are to be regarded as free carboxylate groups in the context of the present invention.

In a preferred embodiment, the matrix polymer in the transdermal therapeutic system does not contain acidic functional groups. In a further preferred embodiment, the matrix polymer in the transdermal therapeutic system contains or consists of neutral polymers, i.e. it contains no acidic and no basic functional groups, where “acidic” and “basic” are to be understood in the sense of the Lewis concept.

In a preferred embodiment, the matrix polymer of the matrix layer comprises linear styrene-butadiene-styrene or styrene-isoprene-styrene block copolymer.

Another suitable group of matrix polymers are acrylate polymers, especially in the form of self-crosslinking acrylic-acid-containing acrylate polymers, which crosslink via the addition of aluminium or titanium compounds with the formation of chelate esters. In such self-crosslinking matrix polymers, the acrylic acid bonded, for example, to the titanium forms crosslinking points. Alternatively, non-self-crosslinking acrylate copolymers, for example those with hydroxy groups or acid groups as functional groups, can be used. Such polymers are commercially available, for example, under the brand name Durotak from Henkel.

Especially suitable acrylate polymers may be terpolymers of 2-ethylhexyl acrylate, vinyl acetate, 2-hydroxyl ethyl acrylate, terpolymers of 2-ethylhexyl acrylate, vinyl acetate and acrylic acid, tetrapolymers of 2-ethylhexyl acrylate, butyl acrylate, vinyl acetate and acrylic acid or a mixture thereof.

Self-crosslinking tetrapolymers of 2-ethylhexyl acrylate, butyl acrylate, vinyl acetate and acrylic acid and self-crosslinking or non-self-crosslinking terpolymers of 2-ethylhexyl acrylate, vinyl acetate and 2-hydroxyl ethyl acrylate are very especially suitable.

According to the above definition, self-crosslinking acrylate polymers containing acrylic acid do not contain free carboxylic acid and/or carboxylate groups, as these are bound via the added crosslinker.

Another polymer that is useful as a matrix polymer is polyisobutylene, which can be used alone or in combination with polybutylene.

Furthermore, polar vinyl polymers such as polyvinylpyrrolidone or polyvinyl alcohol can be used as matrix polymers.

Lastly, non-organic polymers such as polysiloxanes can also be used as matrix polymers.

It is also possible to use mixtures of the aforementioned polymers as matrix polymers, although it must be assumed, by way of limitation, that the polymers are sufficiently compatible with each other so that there is no substantial segregation of the polymer components. However, due to the higher processing effort required for the production of matrix layers based on different polymers, it is preferred if the TTS contains only one polymer type as a matrix layer. Furthermore, due to the ease of processing, it is preferred that the matrix polymer consists of only one (or one each) of the mentioned polymers.

The matrix polymer makes up the largest proportion in the matrix layer. For example, the matrix layer usually contains a proportion of matrix polymer in the range of 70 to 99% by weight preferably 75 to 97% by weight, and very especially preferably 80 to 95% by weight.

In addition to the components already mentioned, the matrix layer can also contain common additives. The type of possible additives depends on the used polymer and the active substance. According to their function, they can be divided into plasticisers, tackifiers, stabilisers, carriers, diffusion- and penetration-regulating additives or fillers. The physiologically harmless substances that come into question for this purpose are known to the person skilled in the art.

The matrix layer has such an inherent tack that permanent contact with the skin is ensured.

Examples of suitable plasticisers include diesters of dicarboxylic acids, for example di-n-butyl adipate, and triglycerides, especially medium-chain (i.e. C₆-C₁₄) triglycerides, for example the caprylic/capric acid of coconut oil.

Abietyl alcohol, for example, can be used as a suitable tackifier.

In one embodiment, it is preferred that the transdermal therapeutic system according to the invention does not contain silicon dioxide as a permeation-promoting additive, and it is especially preferred if the transdermal therapeutic system according to the invention does not contain added silicon dioxide.

Preferably, the active-substance-impermeable backing layer is constructed from a composite material and comprises an aluminised film. The film is based expediently on an active-substance-impermeable material, with polyesters such as polyethylene terephthalate, polybutylene terephthalate, polyethylene naphthalate, polyolefins such as polyethylene or polypropylene, ethylene vinyl acetate, polyvinyl chloride, polyamide (nylon) or polyurethane being potential suitable materials.

The active-substance-impermeable backing layer may also have a protruding end relative to the underlying active-substance-containing layer, with the area of the protruding end being coated with an adhesive that does not contain any active substance. This design minimises accidental contamination of other body sites by the wearer.

For example, the removable protective layer in contact with the matrix layer and removed prior to use comprises the same materials used to produce the backing layer, provided these are made removable, such as by a silicone treatment. Other removable protective layers include, for example, polytetrafluoroethylene, treated paper, cellophane, polyvinyl chloride and the like. If the laminate according to the invention is divided into formats (patches) suitable for therapy before the protective layer is applied, the protective layer formats then to be applied can have a protruding end, with the aid of which they can be more easily removed from the patch.

The application time for which the transdermal therapeutic system is intended and designed is preferably at least 12 hours, further preferably at least 24 hours and even more preferably at least 48 hours. The amount of active substance is to be adjusted according to the desired application time.

Preferably, the transdermal therapeutic system according to the invention described herein is designed to achieve a daily dose of delivered phenprocoumon in the range of about 0.5 to 5 mg, and preferably of 1 to 3 mg. For this purpose, the TTS is formed in a suitable size, for example in the range of 15 to 40 cm² (in relation to the contact area of the adhesive matrix layer).

Further or alternatively, the transdermal therapeutic system according to the invention described herein is configured to achieve a cumulative active substance flux in 24 h of 0.005 to 0.2 mg/1.16 cm² area of the active-substance-containing matrix layer. Preferred is a flux in the range of 0.03 to 0.1 mg/1.16 cm² and especially preferred is a flux of 0.05 to 0.09 mg/1.16 cm².

The transdermal therapeutic system according to the invention is suitable for treating patients with thrombosis or atrial fibrillation. Therefore, a further aspect of the present invention relates to a transdermal therapeutic system as described above for therapeutic or preventive treatment of vaso-occlusive blood clots, especially thrombosis or atrial fibrillation, and usually preferably for long-term thrombosis prophylaxis. A still further aspect of the present invention relates to a transdermal therapeutic system as described above for therapeutic or preventive treatment of patients after implantation of artificial heart valves and/or heart support systems and/or artificial vascular bypasses. For this therapeutic or preventive treatment, patients who develop coumarin necrosis during treatment with phenprocoumon or who are at increased risk of such side effects, such as women with obesity, in whom therapy is started after the onset of menopause or immediately after the birth of a child, are expediently excluded. If it should be shown that these side effects are reduced with the transdermal administration of phenprocoumon, for example because a more constant level of phenprocoumon can be set in the body, this would be a further advantage of the transdermal therapeutic systems according to the invention over conventional oral administration.

The present invention additionally also relates to a method for the therapeutic or preventive treatment of patients benefiting from or in need of treatment with anticoagulants, wherein the treatment includes applying to the skin of the patient a transdermal therapeutic system as described above. Preferably, the patient is a patient with thrombosis or atrial fibrillation, or a patient at risk of thrombosis or atrial fibrillation, or a patient in whom an artificial heart valve and/or heart support systems and/or an artificial vascular bypass has been implanted.

Lastly, the present invention relates to a method for producing the transdermal therapeutic system described above, comprising at least the following steps:
applying a solution comprising the matrix polymer, phenprocoumon and at least one pharmaceutically acceptable solvent to a removable protective layer,
drying the solution with formation of an adhesive matrix layer, and
applying an active-substance-impermeable backing layer to the adhesive matrix layer.

The pharmaceutically acceptable solvent comprises conventional solvents used for pharmaceutical applications, such as toluene or ethyl acetate, as well as mixtures of such solvents.

With regard to the advantages of the method for producing the transdermal therapeutic system described above, please refer to the comments regarding the transdermal therapeutic system.

The invention will be explained in greater detail with reference to the exemplary embodiments described below.

### EXAMPLE 1: DETERMINATION OF THE SATURATION CONCENTRATION C_{S} OF PHENPROCOUMON IN VARIOUS POLYMERS

The saturation concentration Cₛ of phenprocoumon was determined in various polymer matrices according to the method of Liu (Liu, P., Gargiulo, P., Wong, J., and Novartis (1997). A Novel Method for Measuring Solubility of a Drug in an Adhesive. Pharmaceutical Research 14, p. 317).

In this method, known among experts as the “sandwich” method, the saturation concentration is determined as follows:

A laminate is built up with the following sequence of layers: protective film—donor layer with active substance (dissolved and undissolved)—active-substance-permeable membrane—acceptor layer without active substance—protective film. The two protective films consist of identical material; the matrix material of the donor layer and acceptor layer is also identical.

The donor layer is prepared by dissolving the active substance in a solution of the polymer in organic solvent. In this process, the concentration of the active substance must be chosen to be high enough that an undissolved residue is visible in the polymer matrix, so that the saturation concentration Cₛ in the donor layer is safely exceeded. This solution is spread on the protective film and the process solvent is evaporated. Then, the adhesive surface of the donor layer is covered with the membrane. The membrane used is a dialysis tube made of regenerated cellulose (ZelluTrans, from Roth, 46 mm flat width), which has been cut open lengthwise. The acceptor layer is produced analogously to the donor layer without active substance and is applied to the other side of the membrane.

The laminates produced in this way are then stored at room temperature for 7 days, during which diffusion of the active substance through the membrane into the acceptor layer occurs. Subsequently, the active substance concentration in the donor layer is determined. For this purpose, aliquots of approx. 1 cm² are punched using a punching tool with a standardised surface area. The membrane is then removed, the punched pieces without membrane are weighed, and their weight is documented (m₁). Then, the punched pieces are placed in organic solvent to dissolve the matrices. The backing layers are removed, washed and dried and their weight (m₂) is determined. From both measured values, the weight of the polymer portion of the acceptor layer m₃ is obtained as follows:

m₃=m₁−m₂

Subsequently, the concentration of phenprocoumon is determined in the solution using HPLC, and its concentration in the donor layer is calculated. The saturation concentrations of phenprocoumon in different polymer matrices determined according to this experimental approach are summarised in Table 1:

TABLE 1
Cₛ of phenprocoumon in different polymer matrices

| Polymer | Cₛ of | Solvent used |
|---|---|---|
| | phenprocoumon | to dissolve |
| | [%] | the polymer |
| Polyisobutylene | 2.8 | Toluene |
| Styrene-isoprene-styrene block copolymer | 3.9 | Toluene |
| Acrylate copolymer of 2-ethylhexyl | 6.11 | Ethyl acetate |
| acrylate, vinyl acetate and 2-hydroxyl | | |
| ethyl acrylate | | |
| Acrylic copolymer of 2- | 7.26 | Ethyl acetate |
| ethylhexyl acrylate, butyl | | |
| acrylate, vinyl acetate, acrylic | | |
| acid | | |
| Polysiloxane | 0.3 | Ethyl acetate |

Table 1 shows that the saturation concentration Cₛ of phenprocoumon in neutral polymers is about >5%. Slightly higher saturation concentrations were determined in acidic polymers, which can be explained by the vinylogous acid group of the active substance. A especially low Cₛ of phenprocoumon was measured in polysiloxane.

### EXAMPLE 2: PRODUCTION OF PHENPROCOUMON TTS

Transdermal therapeutic systems based on different base polymers were produced:

a) TTS with Polyisobutylene (PIB)

Production of Polyisobutylene Solution

50 g each of Oppanol B 10 and Oppanol B 100 are dissolved in 250 g toluene with stirring for several days. 350 g of a solution with 28.6% solids are obtained.

Production of Samples 1, 2 and 3

In each 100 g of the produced polyisobutylene solution, 0.6 g, 0.9 g and 1.2 g phenprocoumon, respectively, are sprinkled in with stirring; stirring is continued for several hours until the solids are completely dissolved. These three solutions are spread using an Erikson squeegee onto a siliconised 100 μm PET film (Mitsubishi RN 100). After evaporation of the toluene, the basis weight is about 90 g/m². The phenprocoumon concentration in sample 1 is about 2%, that in sample 2 is about 3%, and that in sample 3 is about 4%.

b) TTS with Styrene-Isoprene-Styrene (SIS)

Production of Styrene-Isoprene-Styrene Block Copolymer Solution

95 g styrene-isoprene-styrene block copolymer and 5 g abietyl alcohol are dissolved in 250 g toluene by stirring for several days. 350 g of a solution with 28.6% solids are obtained. Since styrene-isoprene-styrene block copolymer is not adhesive, abietyl alcohol is added as a tackifying resin.

Production of Samples 4, 5 and 6

In each 100 g of the produced styrene-isoprene-styrene block copolymer solution, 0.8 g, 1.2 g and 1.5 g phenprocoumon, respectively, are sprinkled with stirring; stirring is continued for several hours until the solids are completely dissolved. These three solutions are spread using an Erikson squeegee onto a siliconised 100 μm PET film (Mitsubishi RN 100). After evaporation of the toluene, the basis weight is about 90 g/m². The phenprocoumon concentration in sample 4 is about 2.7%, that in sample 5 is about 4%, and that in sample 6 is about 5%.

c) TTS with Polyacrylates

Polyacrylates used as medical adhesives can be obtained commercially as solutions in organic solvents. For samples 7 to 9, the trade products from Henkel, Durotak 87-4287—a neutral acrylate copolymer of 2-ethylhexyl acrylate, vinyl acetate and 2-hydroxyl ethyl acrylate in ethyl acetate (39% solids content)—and Durotak 387-2051, an acidic acrylate copolymer of 2-ethylhexyl acrylate, butyl acrylate, vinyl acetate, acrylic acid in ethyl acetate/n-heptane (51.5% solids content) was used as a reference.

TTS in Neutral Polyacrylate Samples 7, 8 and 9

In each 100 g of Durotak 87-4287, 2 g, 3 g and 4 g phenprocoumon, respectively, are sprinkled with stirring; stirring is continued for several hours until the solids are completely dissolved. These three solutions are spread using an Erikson squeegee onto a siliconised 100 μm PET film (Mitsubishi RN 100). After evaporation of the solvent, the basis weight is about 60 g/m². The phenprocoumon concentration in sample 7 is about 4.9%, that in sample 8 is about 7.1%, and that in sample 9 is about 9.3%.

TTS in Acid Polyacrylate Samples 10, 11 and 12

In each 100 g of Durotak 387-2051, 2 g, 4 g and 6 g phenprocoumon, respectively, are sprinkled with stirring; stirring is continued for several hours until the solids are completely dissolved. These three solutions are spread using an Erikson squeegee onto a siliconised 100 μm PET film (Mitsubishi RN 100). After evaporation of the solvent, the basis weight is about 90 g/m². The phenprocoumon concentration in sample 7 is about 3.8%, that in sample 11 is about 7.2%, and that in sample 12 is about 10.4%.

d) TTS in Polysiloxane

Production of the Solution of Polysiloxane in Toluene

Phenprocoumon is sufficiently soluble in aromatic hydrocarbons, but not in n-heptane. Since toluene polysiloxane solution is not commercially available, BIO PSA 4201 from Dow Chemicals (polysiloxane in n-heptane) was used as starting material. The solvent was evaporated off and the rubbery polymeric residue was dissolved with enough toluene to obtain a solution with approx. 75% solids.

Production of Samples 13, 14 and 15

In each 100 g of the produced polysiloxane solution, 0.25 g, 0.5 g and 1 g phenprocoumon, respectively, are sprinkled with stirring; stirring is continued for several hours until the solids are completely dissolved. These three solutions are spread using an Erikson squeegee onto a siliconised 100 μm PET film (Mitsubishi RN 100). After evaporation of the toluene, the basis weight is about 90 g/m². The phenprocoumon concentration in sample 13 is about 0.33%, that in sample 14 is about 0.66%, and that in sample 15 is about 1.3%.

Crystallisation of the active substance occurred in samples 14 and 15.

Permeation Results

Permeation experiments were carried out with samples 1 to 15 in a Franz cell with human skin. The experimental parameters are summarised in Table 2.

TABLE 2
Test parameters for in vitro permeation

| | | | | Permeation time |
|---|---|---|---|---|
| Permeation | Punched | Acceptor | Water bath | Thickness of the |
| area | part area | medium | temperature | skin |
| About 1.6 cm² | 1.16 | 10 ml | 32° C. | 24 hours/ |
| | | physiological | | Approx. 500 μm |
| | | saline solution | | |

Table 3 shows the results of the permeation studies, the absolute contents of phenprocoumon and the active substance utilisation.

TABLE 3
Mean (x out of n = 6) phenprocoumon flux, measured on human skin 500 pm
in Franz cells over 24 hours.

| | Content of | | |
|---|---|---|---|
| Sample | phenprocouman | Cumulative flux in 24 h | Active substance |
| No./Polymer | [mg/1.16 cm²] | [mg/24] | utilisation [%] |
| 1 PIB | 0.14 | 0.006 | 4.3 |
| 2* PIB | 0.21 | 0.063 | 30 |
| 3 PIB | 0.28 | 0.071 | 25 |
| 4 SIS | 0.19 | 0.003 | 1.6 |
| 5* SIS | 0.28 | 0.057 | 20 |
| 6 SIS | 0.35 | 0.066 | 19 |
| 7 neutral PA | 0.34 | 0.008 | 2.4 |
| 8* neutral PA | 0.49 | 0.073 | 14.9 |
| 9 neutral PA | 0.68 | 0.086 | 12.6 |
| 10 acidic PA | 0.4 | 0.008 | 2 |
| 11 * acidic PA | 0.75 | 0.054 | 7.2 |
| 12 acidic PA | 1.09 | 0.063 | 5.8 |
| 13*Polysiloxane | 0.04 | 0.009 | 22.5 |
| 14 Polysiloxane | 0.07 | 0.026 | 37.1 |
| 15 Polysiloxane | 0.13 | 0.039 | 36.2 |

* Phenprocoumon concentration close to the saturation concentration Cₛ
Flux rates around about 0.065 mg/1.16 × 24 h enable TTS with an area of about 40 cm², which is of significant advantage for patient compliance.

Table 3 shows that the use of neutral polymer TTS with active areas around 40 cm² makes phenprocoumon available transdermally, with the application of one TTS/day, in daily doses corresponding to the oral daily doses. Patterns 2, 3, 5, 6, 8 and 9 are especially suitable.

With sample 12, a TTS with an area of about 40 cm² can indeed also be obtained, but the active substance utilisation is low at just <6%.

1. A transdermal therapeutic system for the cutaneous administration of phenprocoumon, comprising an active-substance-impermeable backing layer, an adhesive matrix layer and optionally a removable protective layer, characterised in that the adhesive matrix layer contains phenprocoumon with a content ≤12% by weight and at least one matrix polymer.

2-15. (canceled)

16. A transdermal therapeutic system for the cutaneous administration of phenprocoumon, comprising an active-substance-impermeable backing layer, an adhesive matrix layer and optionally a removable protective layer, characterised in that the adhesive matrix layer contains phenprocoumon with a content of 0.5 to ≤12% by weight and at least one matrix polymer, and in that the matrix polymer consists of neutral polymers.

17. The transdermal therapeutic system according to claim 16, characterised in that the content of phenprocoumon in the matrix polymer is 0.5 to 7.5% by weight.

18. The transdermal therapeutic system according to claim 16, characterised in that the phenprocoumon is present dissolved in the matrix polymer.

19. The transdermal therapeutic system according to claim 16, characterised in that the matrix polymer comprises or consists of linear styrene-butadiene-styrene block copolymer or styrene-isoprene-styrene block copolymer.

20. The transdermal therapeutic system according to claim 16, characterised in that the matrix polymer contains or consists of self-crosslinking or non-self-crosslinking acrylate copolymer.

21. The transdermal therapeutic system according to claim 16, characterised in that the matrix polymer contains or consists of polyisobutylene or polybutylene and polyisobutylene.

22. The transdermal therapeutic system according to claim 16, characterised in that the matrix polymer contains or consists of polyvinylpyrrolidone or polyvinyl alcohol.

23. The transdermal therapeutic system according to claim 16, characterised in that the matrix polymer contains or consists of polysiloxane.

24. The transdermal therapeutic system according to claim 16, characterised in that the active-substance-impermeable backing layer is constructed from a composite material and comprises an aluminised film.

25. The transdermal therapeutic system according to claim 16, characterised in that it is designed for an application time of at least 24 hours.

26. The transdermal therapeutic system according to claim 16, characterised in that it is designed to deliver a daily dose of phenprocoumon of about 0.5 to 10 mg.

27. The transdermal therapeutic system according to claim 16 for therapeutic or preventive treatment of vaso-occlusive blood clots, thrombosis or atrial fibrillation.

28. The transdermal therapeutic system according to claim 16 for therapeutic or preventive treatment of patients after implantation of artificial heart valves and/or heart support systems and/or artificial vascular bypasses.

29. A method for producing a transdermal therapeutic system according to claim 16, characterised by the following steps:
applying a solution comprising the matrix polymer, phenprocoumon and at least one pharmaceutically acceptable solvent to a removable protective layer,
drying the solution with formation of an adhesive matrix layer, and
applying an active-substance-impermeable backing layer to the adhesive matrix layer.

30. The transdermal therapeutic system according to claim 16, characterised in that the content of phenprocoumon in the matrix polymer is 2.5 to 7.3% by weight.

31. The transdermal therapeutic system according to claim 16, characterised in that the matrix polymer contains or consists of a terpolymer of 2-ethylhexyl acrylate, vinyl acetate and 2-hydroxyl ethyl acrylate.

32. The transdermal therapeutic system according to claim 16, characterised in that it is designed to deliver a daily dose of phenprocoumon of about 1 to 5 mg.

33. The transdermal therapeutic system according to claim 16 for therapeutic or preventive treatment of long-term thrombosis prophylaxis.
